(19) United States
(12) Patent Application Publication
(10) Pub. No.: US 2019/0076670 A1
(43) Pub. Date: Mar. 14, 2019

(54) Flashing Light Therapy with Image Presentation and Interaction for Treatment of Alzheimer's Disease and Dementia

(71) Applicant: ALZHEIMER'S LIGHT LLC, Miami, FL (US)

(72) Inventor: Andrey Vyshedskiy, Boston, MA (US)

(21) Appl. No.: 16/128,487

(22) Filed: Sep. 11, 2018

Related U.S. Application Data
(60) Provisional application No. 62/606,112, filed on Sep. 11, 2017.
(60) Provisional application No. 62/606,476, filed on Sep. 25, 2017.
(60) Provisional application No. 62/761,615, filed on Apr. 2, 2018.

(51) Int. Cl. A61N 5/06; H05B 41/34

(52) U.S. Cl. CPC A61N 5/0618; H05B 41/34; G16H 20/70; A61N 2005/0627; A61N 2005/0629

(57) ABSTRACT

The flashing light and cognitive therapy or video display presented on a mobile device or another screen used together to treat Alzheimer's disease and improve the brain's function.

### RELATED APPLICATIONS

This application claims the benefit of priority under 35 USC 119(e) to U.S. Provisional Application No. 62/606,112, filed on Sep. 11, 2017, U.S. Provisional Application No. 62/606,476, filed on Sep. 25, 2017, and U.S. Provisional Application No. 62/761,615, filed on Apr. 2, 2018, which are incorporated herein by reference in their entirety.

### FIELD OF THE INVENTION

This invention pertains generally to improving brain functioning and to treating, slowing down, an reducing the symptoms of Alzheimer's disease and dementia.

### BACKGROUND

Flashing light therapy reduces the risk of toxic beta amyloid proteins in the brain. Light flashing 40 times per second for an hour results in a significant reduction in beta amyloid in the next 12-24 hours in the parts of the brain responsible for memory, the hippocampus, in addition to the part of the brain responsible for vision. The flashing light therapy may be used to treat dementia and Alzheimer's disease, an affliction affecting millions of people and their families around the world.

Cognitive training challenges (for example, Sudoku puzzles, memory games, spatial orientation games, crossword puzzles, etc.) also slow down the development and reduce the symptoms of Alzheimer's disease and dementia.

### BRIEF SUMMARY

Some embodiments of this invention provide both the benefits of the flashing light therapy and of video or of cognitive training challenges presented on a mobile or stationary device screen to treat patients with Alzheimer's disease and dementia.

The flashing light and cognitive therapy or video display presented on a mobile device or another screen used together to treat Alzheimer's disease and improve the brain's function better than any of these therapies alone.

A patient should be motivated to stare at a flickering light. Movies and other content presented as a video stream is one way of providing this motivation to keep a patient looking at a screen.

Another possible way of providing the motivation to keep looking at a screen are cognitive activities, such as Sudoku puzzles, memory games, crossword puzzles, action games, spatial orientation games, reading books, etc. They assure that a patient is looking at the flickering display. or a flickering light source next to it. or around it and thus receives the flashing light therapy with open eyes. Furthermore, the cognitive activities promote the natural 40 Hz gamma waves generated by the patient's own neural networks. The persistence of these natural 40 Hz gamma waves is one goal of the combined light and cognitive therapy as these waves activate critical cleaning cells in the brain that eliminate pathologic beta amyloid protein.

The flashing light, for example, at about 40 Hz, may be generated by a LED frame or another light source next to or around the screen within the field of vision of a person viewing the screen or provided by the screen itself next to or within the images observed by the user.

However, many modern displays have display refresh rate of about 60 Hz. On these devices with 60 Hz refresh rate, alternating a single black frame and a single white provides the frequency of flickering light at 30 Hz. However, an LED frame installed around a screen, converts the screen into a combined light and cognitive therapy (or video) delivery device. The computer screen presents cognitive training exercises or videos while the LED frame provides 40 Hz light therapy.

An example method comprises: every t seconds showing on at least a portion of a screen a fixed or changing lighter image for x % of the t seconds and a fixed or changing darker image or color for y % of the t seconds, wherein the lighter image is on average more bright than the darker image or color; wherein t is 1/f; wherein f is between 30 Hz and 50 Hz; wherein x is greater than 0% and less than 100%; and wherein y is greater than 0% and less than 100%.

In some embodiments, f is 40 Hz; x is 33%; and y is 66%.

In some embodiments, f is 40 Hz; x is 66%; and y is 33%.

In some embodiments, f is 40 Hz; x is 50%; and y is 50%.

In some embodiments, the screen is a TV screen.

In some embodiments, screen is connected to or is a part of a computer device.

In some embodiments, the computer device is capable of receiving the user's input.

In some embodiments, the at least a portion of the screen is the entire screen.

In some embodiments, the lighter image allows user to engage in cognitive training or in cognitive activity.

In some embodiments, the lighter image is at least one of: a puzzle, a Sudoku puzzle, a memory game, a crossword puzzle, an action game, a spatial orientation game, a text, or a video.

Another example method comprises: every t seconds showing on a first at least a portion of a first screen a lighter image or color for x % of the t seconds and a darker image or color for y % of the t seconds; and showing an image allowing the user to engage in cognitive training or in cognitive activity on a second at least a portion of a second screen, wherein the first at least a portion of the first screen is adjacent or the second at least a portion of the second screen; wherein t is 1/f; wherein f is between 30 Hz and 50 Hz; wherein x is greater than 0% and less than 100%; and wherein y is greater than 0% and less than 100%.

An example device comprises: a light source attachable to a screen of a computer device capable of presenting cognitive activities to the user; and a controller connected to the light source, wherein the controller is capable of making the light source flash at a frequency between 30 Hz and 50 Hz; and wherein the device does not obscure at least a portion of the screen where the cognitive activities are presented.

In some embodiments, the light source is attached to the screen.

In some embodiments, the light source forms more than 50% of a perimeter of the at least a portion of the screen where the cognitive activities are presented.

In some embodiments, the cognitive activity is at least one of: a puzzle, a Sudoku puzzle, a memory game, a crossword puzzle, an action game, a spatial orientation game, a text, or a video.

Another example device comprises: a light source attachable to a book without obscuring a page within the book; and a controller connected to the light source, wherein the controller is capable of making the light source flash at a frequency between 30 Hz and 50 Hz.

Some embodiments are powered by at least one battery, via a USB connector, or from a power outlet.

In some embodiments, the light source is a frame.

Some embodiments further comprise at least one hook or a clip for attachment to the book.

In some embodiments, the light source comprises at least one LED.

Yet another example device comprises: a light source attachable to eyeglasses; and a controller connected to the light source, wherein the controller is capable of making the light source flash at a frequency between 30 Hz and 50 Hz.

In some embodiments, the light source is attached to the eyeglasses.

In some embodiments, the light source forms more than 50% of a perimeter of the eyeglasses.

Some embodiments are powered by at least one battery.

In some embodiments, the light source is a frame.

Some embodiments further comprise at least one hook or a clip for attachment to the eyeglasses.

Yet another example device comprises: a light source attachable to a virtual reality headset; and a controller connected to the light source, wherein the controller is capable of making the light source flash at a frequency between 30 Hz and 50 Hz.

In some embodiments, the light source is attached to the virtual reality headset.

In some embodiments, the light source is shaped as a frame.

In some embodiments, the light source comprises at least one LED.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE SEVERAL VIEWS OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 shows an LEDs embedded into a frame hanged over a display showing cognitive puzzles or videos.

FIG. 2 shows a light source generating 40 Hz flashing light powered by USB port installed on one side of a display with cognitive puzzles or videos.

FIG. 3 illustrates how 40 Hz light flashing is provided by presenting one light frame followed by two darker frames at 120 frames per second refresh rate.

All illustrations of the drawings are for the purpose of describing selected versions of the present invention and are not intended to limit the scope of the present invention.

### DETAILED DESCRIPTION

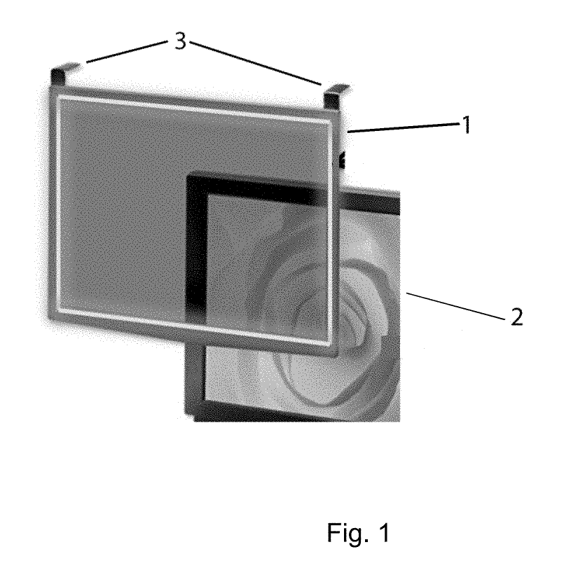

FIG. 1 shows an LED frame 1 (with embedder LEDs) mounted around a display 2 that shows cognitive puzzles or videos. The display 2 may be a computer screen, a portable computer device screen, or an electronic paper display, or a paper book, or any other medium capable of presenting cognitive puzzles or videos. The LED frame comprises at least one LEDs flickering at 40 Hz. It may be powered by USB computer port or by an external power outlet or by a battery. Other light sources, such as light bulbs of suitable size, may be used instead of the LED.

The LED frame may be glued to a display, hanged over a display using hooks or hangers 3, or simply placed over a horizontal display. The LED frame does not have to go around the entire screen.

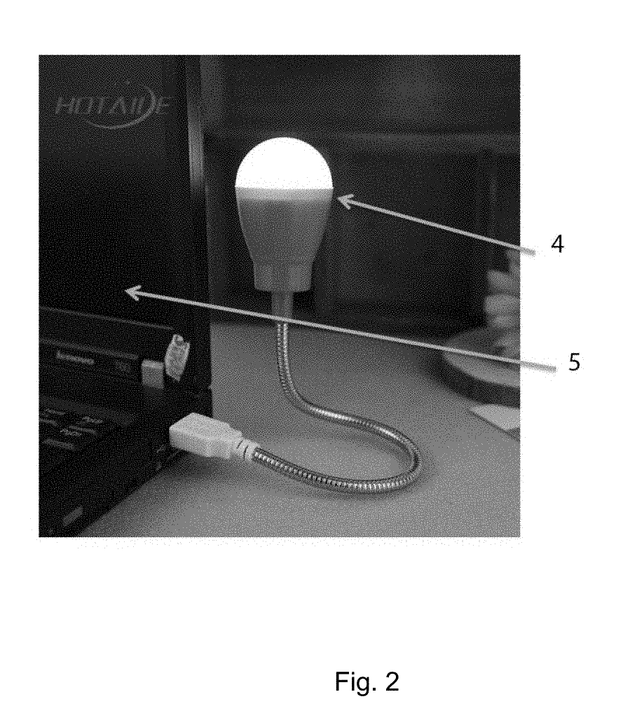

The LED frame may be of any suitable shape: square, circular, or any other form as long as it does not obscure the cognitive puzzles or videos. As shown in FIG. 2, the LED light 4 (powered through a USB port of the computer) source may be positioned near, in front, on the side, above, below, or behind a display 5 showing cognitive puzzles or videos.

Just like on the display, a flashing light frame may also be installed on eyeglass frames or eyeglasses, inside or outside, without entirely obscuring the view. A flashing light frame may also be installed inside a virtual head set without obscuring the view.

The cognitive activities may include Sudoku puzzles, Tic-Tac-Toe games, chess, checkers, memory games, crossword puzzles, action games, spatial orientation games, reading books, watching videos, or any other cognitive activities.

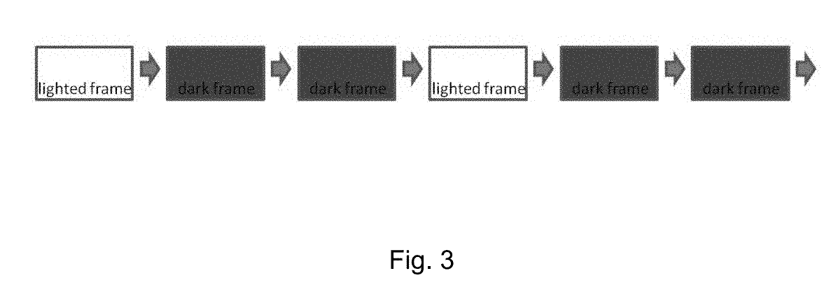

Many modern mobile devices can generate flashing light at 40 Hz. For example, an iPad released in 2017 has 120 Hz refresh rate. A flashing light effect at 40 Hz may be generated by displaying one lighter image frame followed by two darker image frames, as shown in FIG. 3. Alternatively, the image may be shown only in some frames, other frames being uniform in appearance. The lighter and darker are relative measures of how much or how little cumulative light is emitted by an image frame. Such flashing does not bother the viewer because flickering at the rates of 16 Hz or more is not visible.

Another alternative is to display two lighter frames followed by one darker frame at 120 Hz refresh rate. Yet another alternative is to display one lighter frame followed by one darker frame at 80 Hz refresh rate.

The image frames on the screen present engaging cognitive games (such as Sudoku puzzles, memory games, crossword puzzles, action games, spatial orientation games, reading books, etc.) or videos, and the patient undergoes flashing light therapy by looking at the flickering screen.

The example iOS Swift code may be used to present one lighter frame followed by one darker frame at 80 Hz refresh rate:

import UIKit
class ViewController: UIViewController {
@IBOutlet weak var timerText: UITextField!
var st: UInt = 0;
@IBOutlet var viewBackgrounf: UIView!
override func viewDidLoad( ) {
super.viewDidLoad( )
// Do any additional setup after loading the view, typically from a nib.
createDisplayLink( );
print(UIScreen( ).maximumFramesPerSecond);
//viewBackgrounf.backgroundColor = UIColor.red;
}
override func didReceiveMemoryWarning( ) {
super.didReceiveMemoryWarning( )
// Dispose of any resources that can be recreated.
}
func createDisplayLink( ) {
let displaylink = CADisplayLink(target: self,
selector: #selector(step))
displaylink.preferredFramesPerSecond = 80;
displaylink.add(to: .current, forMode: .defaultRunLoopMode);
}
var idx: Int = 0;
func step(displaylink: CADisplayLink) {
idx %= 3;
if (idx == 0) {
viewBackgrounf.backgroundColor = UIColor.black;
}
else {viewBackgrounf.backgroundColor = UIColor.white;}
idx += 1;
}
}

Although the invention has been explained in relation to its preferred embodiment, it is to be understood that many other possible modifications and variations can be made without departing from the spirit and scope of the invention as hereinafter claimed. While this invention has been particularly shown and described with references to preferred embodiments thereof, it will be understood by those skilled in the art that various changes in form and details may be made therein without departing from the scope of the invention encompassed by the appended claims.

1. A method of treatment of Alzheimer's disease in a human user, comprising:
every t seconds showing on at least a portion of a screen a fixed or changing lighter image or color for x % of the t seconds and a fixed or changing darker image or color for y % of the t seconds, wherein the lighter image is on average more bright than the darker image or color;
wherein t is 1/f;
wherein f is between 30 Hz and 50 Hz;
wherein x is greater than 0% and less than 100%; and
wherein y is greater than 0% and less than 100%.

2. The method of claim 1,
wherein f is 40 Hz;
wherein x is 33%; and
wherein y is 66%.

3. The method of claim 1,
wherein f is 40 Hz;
wherein x is 66%; and
wherein y is 33%.

4. The method of claim 1,
wherein f is 40 Hz;
wherein x is 50%; and
wherein y is 50%.

5. The method of claim 1, wherein the screen is a TV screen.

6. The method of claim 1, wherein the screen is connected to or is a part of a computer device.

7. The method of claim 6, wherein the computer device is capable of receiving the user's input.

8. The method of claim 1, wherein the at least a portion of the screen is the entire screen.

9. The method of claim 1, wherein the lighter image or the darker image allows user to engage in cognitive training or in cognitive activity.

10. The method of claim 1, wherein the lighter image or the darker image is at least one of: a puzzle, a Sudoku puzzle, a memory game, a crossword puzzle, an action game, a spatial orientation game, a text, or a video.

11. A method of treatment of Alzheimer's disease in a human user, comprising:
every t seconds showing on a first at least a portion of a first screen a lighter image or color for x % of the t seconds and a darker image or color for y % of the t seconds; and
showing an image allowing the user to engage in cognitive training or in cognitive activity on a second at least a portion of a second screen,
wherein the first at least a portion of the first screen is adjacent or the second at least a portion of the second screen;
wherein t is 1/f;
wherein f is between 30 Hz and 50 Hz;
wherein x is greater than 0% and less than 100%; and
wherein y is greater than 0% and less than 100%.

12. A device for of treatment of Alzheimer's disease in a human user, comprising:
a light source attachable to a screen of a computer device capable of presenting cognitive activities to the user; and
a controller connected to the light source,
wherein the controller is capable of making the light source flash at a frequency between 30 Hz and 50 Hz; and
wherein the device does not obscure at least a portion of the screen where the cognitive activities are presented.

13. The device of claim 12, wherein the light source is attached to the screen.

14. The device of claim 12, wherein the light source forms more than 50% of a perimeter of the at least a portion of the screen where the cognitive activities are presented.

15. The device of claim 12, wherein the cognitive activity is at least one of: a puzzle, a Sudoku puzzle, a memory game, a crossword puzzle, an action game, a spatial orientation game, a text, or a video.

16. The device of claim 12 powered by at least one battery, via a USB connector, or from a power outlet.

17. The device of claim 12, wherein the light source is a frame.

18. The device of claim 12, further comprising at least one hook or a clip for attachment to the screen.

19. The device of claim 12, wherein the light source comprises at least one LED.

20. A device for of treatment of Alzheimer's disease in a human user, comprising:
a light source attachable to a book without obscuring a page within the book; and
a controller connected to the light source,
wherein the controller is capable of making the light source flash at a frequency between 30 Hz and 50 Hz.

21. The device of claim 20 powered by at least one battery, via a USB connector, or from a power outlet.

22. The device of claim 20, wherein the light source is a frame.

23. The device of claim 20, further comprising at least one hook or a clip for attachment to the book.

24. The device of claim 20, wherein the light source comprises at least one LED.

25. A device for of treatment of Alzheimer's disease in a human user, comprising:
a light source attachable to eyeglasses or a virtual reality headset; and
a controller connected to the light source,
wherein the controller is capable of making the light source flash at a frequency between 30 Hz and 50 Hz.

26. The device of claim 25, wherein the light source is attached to the eyeglasses or a virtual reality headset.

27. The device of claim 25, wherein the light source forms more than 50% of a perimeter of the eyeglasses.

28. The device of claim 25 powered by at least one battery.

29. The device of claim 25, wherein the light source is a frame or shaped as a frame.

30. The device of claim 25, further comprising at least one hook or a clip for attachment to the eyeglasses.

31. The device of claim 25, wherein the light source comprises at least one LED.
